(19) United States
(12) Patent Application Publication
(10) Pub. No.: US 2015/0034242 A1
(43) Pub. Date: Feb. 5, 2015

(54) SCAFFOLD AND METHOD OF FORMING SCAFFOLD BY ENTANGLING FIBRES

(71) Applicant: Agency for Science, Technology and Research, Singapore (SG)

(72) Inventors: Jackie Y. Ying, Singapore (SG); Andrew C. A. Wan, Singapore (SG)

(73) Assignee: Agency for Science, Technology and Research

(21) Appl. No.: 14/515,446

(22) Filed: Oct. 15, 2014

Related U.S. Application Data
(62) Division of application No. 11/791,074, filed on May 16, 2007.
(60) Provisional application No. 60/663,872, filed on Mar. 22, 2005.

(51) Int. Cl. B29C 67/20; A61L 27/20; A61L 27/50; B29C 67/24

(52) U.S. Cl. CPC B29C 67/205; B29C 67/246; A61L 27/20; A61L 27/50; B29K 2005/00; B29K 2105/0005
USPC 156/296

(57) ABSTRACT

A porous scaffold is provided, which comprises tangled fibres. A porous scaffold can be formed by applying a fluid to fibres to entangle them. The fibres comprise a polyelectrolyte complex and a cross-linker. The cross-linker links polyelectrolytes within individual fibres and inhibits secondary polyelectrolyte complication between adjacent fibres.

### CROSS-REFERENCE TO RELATED APPLICATIONS

This is a division of U.S. application Ser. No. 11/791,074, filed May 16, 2007, which was the National Stage of International Application No. PCT/SG05/00198, filed Jun. 20, 2005, which claims the benefit of U.S. Provisional Application No. 60/663,872, filed Mar. 22, 2005, the contents of which applications are incorporated herein by reference.

### FIELD OF THE INVENTION

The present invention relates generally to scaffolds, and more particularly to methods of forming scaffolds by entangling fibres.

### BACKGROUND OF THE INVENTION

Porous scaffolds are useful in various fields and industries, including tissue engineering.

There are known techniques of preparing porous scaffolds directly from solutions such as chitosan solutions. For example, an aqueous chitosan solution may be freeze-dried to form a fibrous and porous structure. The porous structure can be immersed in an alkaline solution to be stabilized. Another possible technique is to consolidate fibres with a chemical binder at elevated temperatures.

However, these techniques have some drawbacks. One problem with these techniques is that the scaffold material is subjected to drastic temperature change and chemical treatment, which can have some adverse effects on the properties of the scaffold or some components incorporated in the scaffold. For instance, the porosity and pore size of a scaffold can significantly decrease during drying and it can be difficult to control the porosity and pore sizes of a scaffold formed by a freeze-drying technique. Further, excessive heating or certain chemical treatment can destroy proteins incorporated in a scaffold or their three-dimensional structures, the integrity of which can be important for biomedical scaffolds in many applications.

Accordingly, there is a need for an alternative method of forming scaffolds. There is also a need for a method of forming scaffolds that can overcome one or more of the aforementioned problems.

### SUMMARY OF THE INVENTION

There is provided a porous scaffold comprising tangled fibres. The porous scaffold can be formed by applying a fluid to fibres to entangle them. The fibres comprise a polyelectrolyte complex and a cross-linker. The cross-linker links polyelectrolytes within individual fibres and inhibits secondary polyelectrolyte complexation between adjacent fibres.

Advantageously, the scaffold can be formed without excessive heating or the use of chemical binders, and the porosity and pore sizes of the scaffold can be conveniently controlled.

Therefore, in accordance with an aspect of the present invention, there is provided a method of forming a porous scaffold. The method comprises providing fibres comprising polyelectrolytes forming a polyelectrolyte complex. The fibres further comprise a cross-linker linking the polyelectrolytes within individual fibres for inhibiting secondary polyelectrolyte complexation between adjacent fibres. A fluid is applied to the fibres to entangle the fibres to form a porous structure. The cross-linker may comprise silicon and may link the polyelectrolytes through Si—O bonds. The fibres may be formed from a polyanion solution and a polycation solution by interfacial polyelectrolyte complexation.

In accordance with another aspect of the present invention, there is provided a scaffold formed in accordance with the method described in the preceding paragraph.

In accordance with a further aspect of the present invention, there is provided a porous scaffold comprising tangled fibres. The fibres comprise polyelectrolytes forming a polyelectrolyte complex. The fibres further comprise a cross-linker linking the polyelectrolytes within individual fibres and inhibiting secondary polyelectrolyte complexation between adjacent fibres.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention,

FIG. 1 is a schematic diagram illustrating a process of hydroentanglement, exemplary of an embodiment of the present invention;

FIG. 2 is a schematic diagram illustrating secondary polyelectrolyte complexation between adjacent fibres;

FIG. 3 is a stereomicroscope image of a scaffold, exemplary of an embodiment of the present invention, at a magnification ratio of 150;

FIG. 4 is a scanning electron microscope (SEM) image of the scaffold of FIG. 3;

FIG. 5 is an SEM image of a fibre incorporating silica formed by interfacial polyelectrolyte complexation; and

FIG. 6 is an SEM image of a collagen-modified polyelectrolyte complex fibre incorporating silica.

### DETAILED DESCRIPTION

In a process of forming a scaffold, exemplary of embodiments of the present invention, a fluid such as water is applied to fibres with sufficient pressure to entangle the fibres to form a porous structure. The fibres contain a polyelectrolyte complex (also called polyion complex) and a cross-linker. The polyelectrolyte complex includes a polyanion and a polycation, which are collectively referred to as polyelectrolytes or polyions. The cross-linker can cross-link the polyelectrolytes within a strand of fibre thus inhibiting secondary complexation of polyelectrolytes between adjacent fibres during the entanglement treatment. Secondary complexation of polyelectrolytes is considered inhibited if it is prevented or reduced. The cross-linker can include silicon, which can bind to the polyelectrolytes through Si—O bonds. For example, the cross-linker can include siloxane bonds (Si—O—Si), such as in silica. The fibres used may be prepared in any suitable manner, such as by interfacial polyelectrolyte complexation as will be described below. Additional materials, such as modifiers, may be incorporated into the fibres, as will be further described below.

Advantageously, the porosity and pore sizes of scaffolds so formed are controllable. For example, the porosity may vary from 10% to 98%. It is also not necessary to subject the scaffold material to freezing, heating, or toxic chemical treatment during the formation process.

The fibres may be entangled with a suitable fluid such as water. For example, the fibres may be entangled by hydroentanglement, also conventionally referred to as spunlace, jet entanglement, water entanglement, hydraulic needling, or hydrodynamic needling.

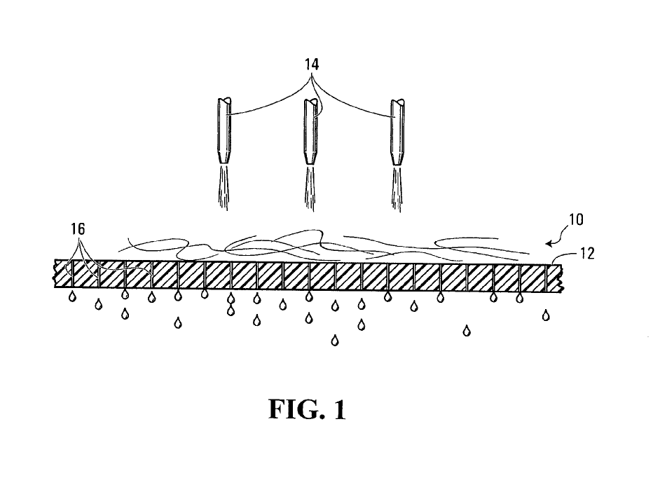

An exemplary hydroentanglement treatment is illustrated in FIG. 1. Loose fibres 10 to be treated are placed on a support 12. The total thickness of fibres 10 may vary. Generally, it may be less than 20 mm. Typically, it may be less than 5 mm. Jets of water are applied to fibres 10, for example, from nozzles 14. While three nozzles 14 are depicted, the number of water jets may vary in different applications. The water jets strike fibres 10 to compact them. The water jets may be needling water jets and strike different spots on fibres 10, creating localized impact. The water streams may be scanned over different areas on fibres 10. To do so, support 12 and nozzles 14 may move relative to each other during treatment. Either one of support 12 and nozzles 14 may be moved while the other remains stable. In industrial production, it may be advantageous to have a continuously movable support, which can also serve as a conveyor in a production line.

Support 12 has small openings 16 through which water, but not fibres 10, may pass. For example, support 12 can be perforated or porous. Each opening 16 may have a diameter of about 200 microns. A screen or a frit may be used as support 12. A frit may be made of a metal plate with a mesh of uniformly distributed openings.

After initial impact, the water passes through fibres 10 and support 12 through openings 16 as indicated by the arrows below support 12. As can be appreciated, accumulation of water around fibres 10 can lessen the impact the water jets on fibres 10.

When the water jets strike fibres 10 at sufficiently high speed or pressure, the impact of the water jets can compact fibres 10 and cause fibres 10 to tangle. High speed water is applied until fibres 10 are sufficiently entangled and compacted to form a stable porous structure. A stable structure can retain its shape and have good stability in water. The duration of applying water can vary depending on the particular application. A person skilled in the art can readily determine the minimum duration required to achieve a desired stability of the resulting scaffold in a particular application.

To create enough impact, the water pressure and flow rate at nozzles 14 should be sufficiently high but may vary depending on the application and a number of factors such as fibre material, fibre size and shape, the desired properties of the resulting scaffold including porosity, pore size and mechanical strength, and the distance from nozzle 14 to fibres 10. Suitable water pressure and flow rates can be readily determined by persons skilled in the art in a given application. The water pressure and flow rate can also be varied during one treatment. For example, the water pressure may be gradually increased as the fibres become more compacted.

The water used may be pre-treated, such as deionized, if appropriate or desirable in a given application. Additives may be added to the water if desired. For example, salt or buffer components may be added to equilibrate the resulting scaffold prior to use in tissue culture applications. Water may also be substituted by another suitable fluid in appropriate situations. For example, a different liquid or even a gas may be used.

As can be appreciated, the external profile of the porous structure can, in part, substantially conform to the shape of the upper surface of support 12. Thus, the lower side of the resulting scaffold can be formed in a desirable shape by providing a corresponding support surface. Further, fibres 10 can be enclosed and confined within a die (not shown) during the hydroentanglement treatment so that the porous scaffold can have an external profile substantially conforming to the inner surface of the die.

Fibres 10 as depicted are loose and unwoven. However, in an alternative embodiment, woven fibres may be used, for example, for controlling the porosity of the formed scaffold.

Different fibres may be entangled together to form scaffolds with different regional properties and characteristics. For example, a scaffold may have different layers for mimicking an in vivo environment.

Fibres may be added during hydroentanglement, such as to an initial layer of fibres before the initial fibres are fully entangled. In this manner, thicker scaffolds may be produced.

In alternative embodiments, the fibres may be subjected to a hydroentanglement treatment different from that shown in FIG. 1. For example, when openings 16 of support 12 are of suitable sizes and distribution, a single stream of water may be applied substantially uniformly to fibres 10. In this case, fibre entanglement can still result because the water flow through fibres 10 at different rates in different regions. In another example, openings 16 may not be necessary if jets of water are applied to the fibres and waste water can be otherwise efficiently removed. In yet another example, water may be applied to the fibres from both sides.

Hydroentanglement techniques conventionally used in the textile industry for consolidating nonwoven webs of fibres may be suitable in some applications. Some suitable conventional hydroentanglement processes are described in U. Munstermann et al. “Hydroentanglement process”, in Nonwoven Fabrics Raw Materials, Manufacture, Applications, Characteristics, Testing processes, edited by W. Albrecht, H. Fuchs, W. Kittelmann, Wiley-VCH: Weinheim, 2000; and U.S. Pat. No. 6,112,385 to Gerold Fleeissner and Alfred Watzl, issued Sep. 5, 2000, the contents of each of which are incorporated herein by reference.

The fibres used in the hydroentanglement treatment may have any suitable size and shape. The average diameters of the fibres may be in the range of tens of microns. The lower limit of the diameter may be dictated by the mechanical properties of the fibres. The upper limit of the diameter may depend on how the particular fibre material can be effectively entangled by hydroentanglement. The lengths of fibres may also vary, depending on the application. For example, the lengths may be in the range of 1 to 1,000 mm.

The fibres may be pre-treated, such as washed, before being entangled. As can be appreciated, wetted fibres can be easier to manipulate than dry fibres.

Fibres 10 can include any polyelectrolyte complex. A polyelectrolyte complex can be formed by two oppositely charged polyelectrolyte molecules, a polyanion and a polycation. A polyelectrolyte is typically a macromolecular species that upon being placed in water or any other ionizing solvent dissociates into a highly charged polymeric molecule. Exemplary polyelectrolyte complexes include alginate-chitosan, heparin-chitosan, chondroitin sulfate-chitin, hyaluronic acid-chitosan, DNA-chitin, RNA-chitin, poly(glutamic acid)-poly(ornithic acid), polyacrylic acid-poly(lysine), and poly(ethyleneimine)-gellan complexes, and the like.

Suitable polyelectrolyte materials for forming polyelectrolyte complexes include natural polyelectrolytes, synthetic polyelectrolytes, chemically modified biopolymers and the like. Exemplary polyelectrolyte materials include carboxylated polymers; aminated polymers such as poly(ethyleneimine); chitin and chitosan and their derivatives; acrylate polymers; nucleic acids such as DNA and RNA; histone proteins; acidic polysaccharides and their derivatives such as chondroitin sulfate, heparin and alginate; poly(amino acids) such as poly(lysine) and poly(glutamic acid); hyaluronic acid; poly(ornithic acid); polyacrylic acid; gellan; and the like. The choice of the polyelectrolyte materials may depend on the application in which the scaffold is to be used and the particular processes employed for forming the fibres. For example, the alginate and chitosan pair may be used in biomedical applications because they have desirable physical, chemical and biochemical properties.

Polyelectrolyte complexes can form when oppositely charged polyelectrolytes are brought close to each other in a process known as interfacial polyelectrolyte complexation. For example, alginate (a polyanion) and chitosan (a polycation) can form a polyelectrolyte complex in such a process. In such a process, a polyanion solution and a polycation solution are brought close to each other, forming an interface. In the interface region, local complexation can occur. Complexation refers to the binding of two oppositely charged polyelectrolytes to form a polyelectrolyte complex. The polyelectrolyte complex formed can become insoluble due to neutralization of charges. Thus, a strand of fibre can be drawn from the interface region and polyelectrolyte complex fibres can be prepared.

The complexation process of forming polyelectrolyte complexes in each fibre is referred to herein as “primary” polyelectrolyte complexation. The polyelectrolyte complexes between adjacent fibres may also form larger complexes through “secondary” polyelectrolyte complexation, particularly when water is introduced into the fibres.

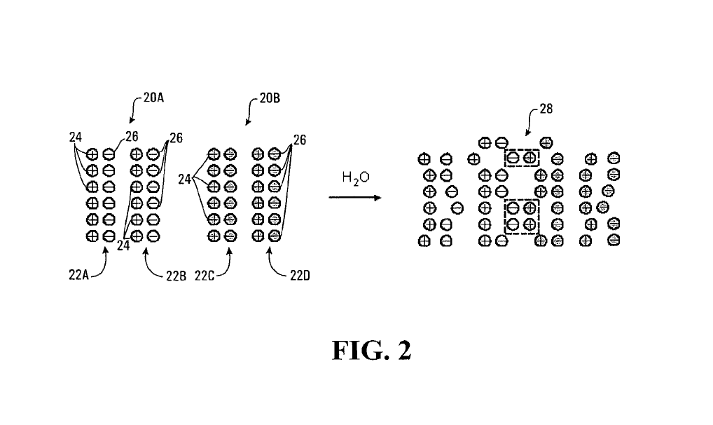

FIG. 2 schematically illustrates the process of secondary polyelectrolyte complexation. Two strands of fibre 20A and 20B are shown. As depicted, each of fibres 20A and 20B includes two polyelectrolyte complexes, 22A and 22B for fibre 20A, and 22C and 22D for fibre 20B, which are formed by primary polyelectrolyte complexation. Polyelectrolyte complexes 22A to 22D are also collectively and individually referred to as complexes 22. While two polyelectrolyte complexes 22 are depicted for each fibre, it should be understood that a fibre may contain different numbers of polyelectrolyte complexes. Each vertical column of circles 24 or 26 represents a polyelectrolyte. Circles 24 represent positively charged groups and circles 26 represent negatively charged groups. Thus, each column of circles 24 represents a polycation and each column of circles 26 represents a polyanion. As shown, each polyelectrolyte complex 22 is formed of a polycation and a polyanion. When fibres 20A and 20B are pressed against each other in water, secondary polyelectrolyte complexation can occur due to the attraction between the oppositely charged groups 24 and 26 from the adjacent fibres. As a result of the secondary polyelectrolyte complexation, a larger polyelectrolyte complex 28 is formed, which holds fibres 20A and 20B together. It should be understood that FIG. 2 is a schematic diagram for illustration purposes only and is not meant to accurately reflect the actual structures of the fibres, the polyelectrolyte complexes, or the polyelectrolytes.

The cross-linker in fibres 10 can be any suitable molecular species that can cross-link the polyelectrolytes within individual fibres for inhibiting secondary electrolyte complexation of the polyelectrolytes between adjacent fibres during the hydroentanglement treatment, thus preventing over-condensation of the fibres by water pressure. The cross-linker may link polyelectrolytes within a single polyelectrolyte complex, between different polyelectrolyte complexes within a fibre, or both. The cross-linker may also link more than two polyelectrolytes together. For example, the cross-linker can include polymeric silica or a siloxane network structure (Si—O—Si). The cross-linker may be formed from a silica precursor having Si—O bonds and free silanol (Si—OH) groups. The silica precursor can be a monomer, oligomer, or polymer. As can be appreciated, secondary polyelectrolyte complexation between adjacent fibres during the entanglement treatment could cause the fibres to bind together so that the resulting scaffold would have low porosity and small pores. When secondary polyelectrolyte complexation between fibres is inhibited, the resulting scaffold can have high porosity and large pores.

A cross-linker such as a silica-containing species incorporated into the fibres can inhibit secondary polyelectrolyte complexation by cross-linking different polyelectrolyte components in each individual fibre. For instance, a silica network can cross-link the polyanions and polycations in a strand of fibre by reacting with the hydroxyl groups of the polyelectrolytes to form Si—O bonds.

Polyelectrolyte complex fibres swell less when the fibres also contain silica. Without being limited to any particular theory, it is believed that the reduction in swelling is due to cross-linking of polyanions and polycations in individual fibres by the silica-containing cross-linker. As can be appreciated, when polyelectrolyte fibres swell, charged ionic groups in the polyelectrolytes may become accessible by other polyelectrolytes. It is thus more likely a polyelectrolyte complex can form between nearby fibres due to the attraction of opposite charges of these charged ionic groups, as illustrated in FIG. 2. During an entanglement treatment, the fibres are pressed against each other, providing a good opportunity for secondary polyelectrolyte complexation between adjacent fibres to occur if it is not inhibited.

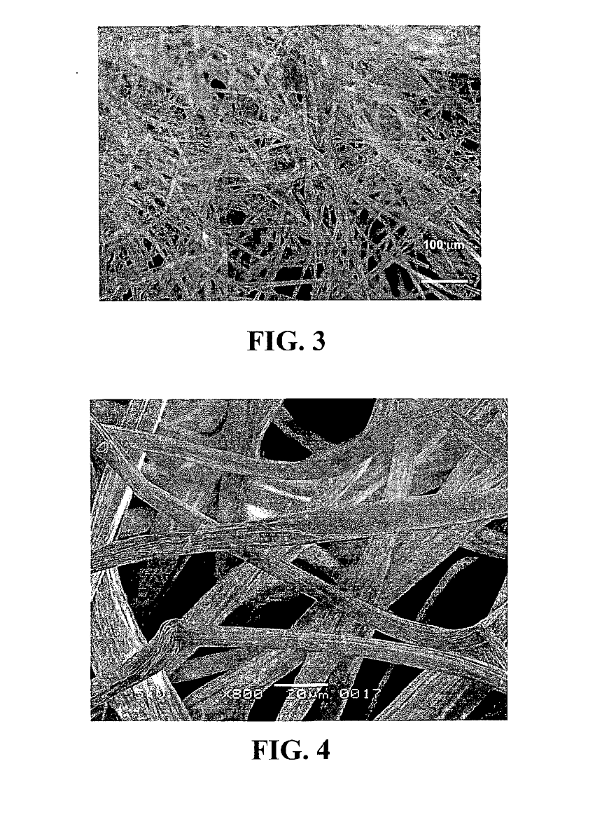

The cross-linker in the fibres can inhibit secondary polyelectrolyte complexation primarily by reducing swelling of the fibres. Again without being limited to any particular theory, when the polyelectrolytes are cross-linked, the fibres swell less in water so that fewer charged ionic groups of the polyelectrolytes are accessible by neighbouring fibres. Further, the cross-linker may also bind to some charged ionic groups, making them unavailable for secondary polyelectrolyte complexation at all. As a result, the formed scaffold can have high porosity and large pore sizes, as illustrated in FIGS. 3 and 4, which show images of a scaffold formed by hydroentangling polyelectrolyte complex fibres incorporating silica, at magnification ratios of 150 and 800 respectively.

The relative amount of the cross-linker in the fibres can be readily determined by persons skilled in the art, depending on the application and the polyelectrolytes used. When the fibres are formed by interfacial polyelectrolyte complexation with alginate and chitosan as the polyelectrolytes and TEOS as the precursor for the cross-linker, the weight ratio of chitosan, alginate and TEOS in the interfacial region can be between about 8:1:0 and about 1:16:19. It may be advantageous if the ratio is from about 8:1:3.7 to about 1:16:9.4. Within a limit, the porosity and pore sizes of the scaffold can be controlled by adjusting the relative amount of the cross-linker in the fibres.

As now can be appreciated, the cross-linker can be any suitable molecular species that can cross-link the polyelectrolytes in the fibres. For example, suitable acrylates, succinimides, carbodiimides, quinones, and the like may be used as cross-linkers or precursors for cross-linkers.

The cross-linker can be incorporated into fibres 10 by dispersing the cross-linker or a precursor of the cross-linker into one of the polyelectrolyte solutions before forming the fibres. While it is possible to add the cross-linker after the fibres have been formed but before hydroentanglement, adding the cross-linker or its precursor during the formation of the fibres can be advantageous. In the latter case, the cross-linker may be better incorporated into the fibres and it is not necessary to separately treat the fibres to add the cross-linker before hydroentanglement.

Fibres 10 may have surface structures and chemical compositions desirable in a given application. For example, for biomedical applications, the fibres may be biocompatible with the cells to be cultured or grown in the scaffold.

Fibres 10 may also include other materials such as modifiers. The modifiers may include an adhesion-enhancing substances for improving the adhesion of certain cells or molecules to the fibres, or suitable proteins, peptides or other biological components, e.g., for cell culturing. An exemplary modifier is polyethylene glycol (PEG) which can modify the surface property of the fibres. As is known, a PEG modified surface can be non-absorptive and can be used to minimize protein adsorption in vivo. Another exemplary modifier is a peptide with an arginine-glycine-aspartate (RGD) motif. As can be understood, a RGD-modified surface can be highly amenable toward cell attachment and proliferation. A further exemplary modifier is collagen, which can also improve cell attachment and proliferation. The modifiers may also include growth factors, drugs, or the like.

The modifiers can be incorporated into fibres 10 by either dispersing them in one or both of the polyelectrolyte solutions, or attaching them to the polyelectrolytes such as by conjugation. Conveniently, polyelectrolytes have many charged sites in solution, such as carboxyl or amino groups, with which the modifiers can conjugate. When a modifier such as a protein bears an electric charge in the solution, it should be dispersed in the similarly charged polyelectrolyte solution to avoid premature formation of complexes. For example, when collagen, which is usually positively charged in solution, is to be included it should be dispersed in the polycation solution. Further, the amount of modifiers should be limited if they can conjugate with the charged groups such as carboxyl or amino groups of the polyelectrolytes so that sufficient charged groups are available for fibre formation. In this regard, the amount of a biological component, such as a biological signal, required in a biomedical scaffold is typically low so that its inclusion will generally not be problematic.

Fibres 10 may be formed with any suitable interfacial polyelectrolyte complexation technique, including conventionally known techniques such as wet spinning techniques, with possible modifications to incorporate the cross-linker and the modifier. The conventional fibre formation techniques are understood and can be readily performed by persons skilled in the art and will not be described in detail herein. Further details of forming fibres by interfacial polyelectrolyte complexation can be found in, for example, Andrew C. A. Wan et al., “Encapsulation of biologics in self-assembled fibers as biostructural units for tissue engineering”, Journal of Biomedical Materials Research, (2004), vol. 71A, pp. 586-595 (“Wan I”); Andrew C. A. Wan et al., “Mechanism of Fiber Formation by Interfacial Polyelectrolyte Complexation”, Macromolecules, (2004), vol. 37, pp. 7019-7025 (“Wan II”); Masato Amaike et al., “Sphere, honeycomb, regularly spaced droplet and fiber structures of polyion complexes of chitosan and gellan,” Macromolecules Rapid Communication, (1998), vol. 19, pp. 287-289; U.S. patent application publication number 2003/0055211 to George A. F. Roberts, published Mar. 20, 2003; and U.S. Pat. No. 5,836,970 to Abhay S. Pandit, issued Nov. 17, 1998, the contents of each of which are incorporated herein by reference.

Briefly, in an exemplary interfacial polyelectrolyte complexation technique, a polyanion solution such as an alginate solution and a polycation solution such as a chitosan solution are brought close to each other, to form an interface therebetween. Complexes of the oppositely charged polyelectrolytes are formed in the interface, which prevent free diffusion between the two solutions. The complexes can be drawn out of the interface, such as upwardly by a pair of forceps or a needle. As the complexes at the interface are withdrawn, further complexation sites become available and more complexes are formed. The complexes are typically insoluble or can become insoluble in the solvent due to neutralization of charges and thus, a fibre can be continuously drawn out of the interface. The fibres drawn can be very thin, for example, having average diameters in the micron range.

The cross-linker such as silica may be incorporated into the fibres by including the cross-linker or its precursor in one of the polyelectrolyte solutions. For example, tetraethyl orthosilicate (TEOS, Si(OC₂H₅)₄, also commonly called tetraethoxysilane) may be included in one of the polyelectrolyte solutions as a precursor for silica. The added TEOS may be hydrolysed in an acetic acid, forming species having Si—OH (or more generally Si—OR, where R is not Si) terminal groups. These species can form polymeric silica (SiO₂) molecular species through polycondensation. For example, sufficient amount of TEOS may be added to one of the polyelectrolyte solutions so that the volume percent (v %) of hydrolyzed silica in the interfacial region is between 0 to about 50 v %. It can be advantageous if the volume percent is from about 17 to about 33 v %. The silica molecular species may have terminal groups in the general form of Si—OR. Polycondensation may occur before, during and after the fibres are formed from the polyelectrolyte solutions. For example, silica condensation can occur when a fibre strand is drawn out of the polyelectrolyte interface and can also occur during subsequent washing, as the pH value in the fibre's environment increases. A silica molecular species having terminal Si—OH groups can react, for example, with hydroxyl groups and/or carboxyl groups present in the polyelectrolytes such alginate and chitosan, to form Si—O bonds. For instance, the silica molecular species may react with the 6-OH of chitosan to form a Si—O—C bond, and with the COOH group of alginate to form a silyl ester (—Si—O—C(O)—). As can be appreciated, the Si—O—C bond is more stable than the silyl ester bond.

As can be appreciated, other silica precursors may be used. For example, it may be possible to replace TEOS by tetramethyl orthosilicate (TMOS), Si(OCH₃)₄.

Advantageously, preparing the fibres through an interfacial polyelectrolyte complexation process does not require freezing or heating, or the use of toxic organic solvents. Further, proteins, cells or other biological components can be conveniently encapsulated in or immobilized on polyelectrolyte complex fibres.

The exemplary embodiments of the present invention are further illustrated by the following non-limiting examples.

Sample polyelectrolyte fibres were formed by interfacial polyelectrolyte complexation. The polyanion solution had about 1 w/v % of alginate. The polycation solution was acetic acid based and contained chitosan and TEOS. The polycation solution was prepared by mixing a chitosan solution and a TEOS solution. The chitosan solution contained about 0.5 w/v % chitosan in a 2 v % acetic acid solution. The TEOS solution was prepared by adding TEOS to a 0.15 M acetic acid (HOAc), with a volume ratio of 1:9 or 9.39 wt %. The TEOS solution was vortexed for about one to two hours until only one phase was observed. As can be appreciated, the TEOS in the solution was hydrolyzed. The vortexed solution was stored at about 4° C. prior to use. The TEOS and chitosan solutions were mixed at a volume ratio of about 1:3. The TEOS in the mixed solution is of 2.35 wt %. For comparison purposes, some polycation solutions with varying TEOS contents were also prepared.

For RGD-modified samples, maleimide-terminated PEG (MAL-PEG-MAL) and RGD peptide were added to the polyanion solution. A 0.35 w/v % MAL-PEG-MAL (3400 Da) solution was prepared in 100 mM sodium phosphate buffer (pH 6.0). About 1 mg of GCGYGRGDSPG peptide was dissolved in 1 mL of the MAL-PEG-MAL solution. The mixture was allowed to react for one hour. About 6.5 mg of cysteine-modified alginic acid were then added to the MAL-PEG-MAL/peptide mixture, and allowed to react overnight at room temperature. The reaction product was dispersed in 1 w/v % alginic acid solution at a volume ratio of about 1:3 to form the modified polyanion solution.

For collagen-modified samples, about 2 w/v % collagen I in 50 mM phosphoric acid was added to the polycation solution at a volume ratio of about 1:4.

The polyelectrolytes contents in the solutions specified above may vary. For example, the alginate may be of about 0.25 to 2 w/v % in the alginate solution; the chitosan may be of about 0.125 to 2 w/v % in the chitosan solution. The particular choice of the content of a polyelectrolyte may depend on its molecular weight, as can be understood by persons skilled in the art.

The sources and particulars of the chemicals used for preparing the above solutions are listed in Table I.

TABLE I

| Chemical | Source | Notable characteristics |
|---|---|---|
| TEOS | Fluka ™, Switzerland | low viscosity, viscosity of |
| alginic acid | Sigma ™ | 250 cps for a 2% solution at |
| | | 25° C. |
| chitosan | Aldrich ™ | high molecular weight |
| | | Brookfield viscosity at |
| | | 800,000 cps |
| acetic acid | Merck ™, Darmstadt, | |
| (analytical | Germany | |
| research grade) | | |
| MAL-PEG-MAL | Nektar Therapeutics ™, | PEG molecular weight is |
| | San Carlos, California | about 3,400 |
| RGD peptide | Peptron ™, Korea. | custom-synthesized |
| Collagen I | isolated from rat skin | |

To form fibres from the polyion solutions, droplets (20 to 120 μL/droplet) of the polyanion solution and the polycation solution were placed close to each other in a Teflon channel about 3 mm in width. The droplets were brought into contact to form an interface region, using a pair of forceps. A fibre strand was drawn from the interface region. The fibre strand was adhered to the arms of a roll-up apparatus rotating at a rate of about 0.833 rev/min, yielding a fibre drawing rate of about 1.25 mm/s. Further details of the roll-up apparatus and the fibre formation process are described in Wan II, supra.

To study the effects of silica in the fibres, some sample fibres were formed with varying TEOS contents in the polycation solution. Modified fibres were formed with the modified polyion solutions.

The sample fibres were examined to determine their morphology and elemental composition, using a JEOL™ JSM-5600 Scanning Electron Microscope (SEM) equipped with an Oxford Instruments™ Electron Dispersive X-ray (EDX) analysis system. The sample fibres were gold-coated for imaging using a JEOL JFC-1200 Fine Coater with a sputter time of 18 seconds and were imaged under high vacuum. For the EDX analysis, the samples were not gold-coated.

Fourier-transform infrared (FTIR) spectra were recorded on a Digilab™ FTS 7000 FTIR spectrometer equipped with a MTEC-300 photoacoustic (PA) detector. The sample fibres were vacuum dried prior to being loaded into the detector. They were then purged with helium in the detector for 15 minutes. The spectra were recorded in the range of 400-4000 cm⁻¹ by the co-addition of 256 scans at a resolution of 4 cm⁻¹. All PA-FTIR spectra were normalized with respect to a carbon black standard. The spectra data were used to identify chemical compositions in the fibres.

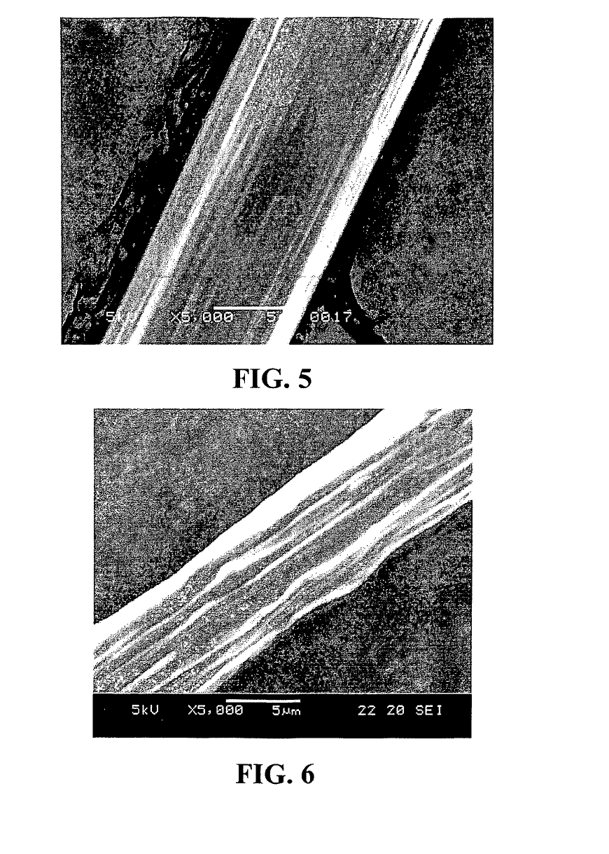

FIG. 5 is an SEM image (at a magnification ratio of 5,000) of a sample fibre containing silica. The presence of silica in the fibre was confirmed by an EDX analysis of the fibre.

FIG. 6 is an SEM image (at a magnification ratio of 5,000) of a collagen-modified fibre.

The swelling abilities of different sample fibres were also measured. The fibres were secured on a glass slide with an adhesive tape. Each fibre to be tested was immersed in about 1 μL of deionized water. The water was allowed to evaporate completely. The fibre diameters were measured with a light microscope before and after swelling. The maximum swelling ratio was calculated as the ratio between the maximum fibre diameter after swelling and the average fibre diameter before swelling. The test results show that the maximum swelling ratio of the sample fibres decreased from about 6.3 to about 3.2 when the hydrolyzed TEOS volume fraction in the polycation solution was increased from zero to about 0.17. Further increase of the TEOS volume did not cause significant change in the maximum swelling ratio.

The sample fibres were dried in air. The dried fibres were washed with deionized water. Typically, fibres and about 1.5 mL of deionized water were placed in a 1.7-mL microcentrifuge tube and allowed to stand for about 5 minutes.

The washed fibres were then subjected to a hydroentanglement treatment on a frit in a die. The die has an internal volume of about 0.5 mL. The total area of the openings in the frit is about 57 mm². Deionized water was passed through the die at a flow rate of 300-350 mL/min for about one minute to entangle the fibres to form a stable scaffold. The flow rate may be increased up to 2000 mL/min.

The water flow rate was then reduced to 5-35 mL/min to wash the formed scaffold for another 5 minutes to remove any residual acid, as well as to allow for complete polycondensation of the silica precursor. Further cross-linking may improve the mechanical properties of the resulting scaffold.

The sample scaffolds were stored in deionized water and then sterilized.

For cell seeding and culturing tests, circular scaffolds of ˜5 mm in diameter were produced.

Sample scaffolds were vacuum dried overnight, and the resulting scaffolds were imaged using a stereomicroscope (Olympus™ SZX stereomicroscope system).

FIGS. 3 and 4 show magnified images of a scaffold formed from samples fibres containing silica as described above.

Comparison of the imaging results shows that sample scaffolds formed from fibres incorporating silica have higher porosity and larger pore sizes than those formed with fibres containing no silica. The porosity of the sample scaffolds is estimated to vary from 10% to 98%. As can be understood by persons skilled in the art, the porosity of a scaffold may be measured using a technique described in, for example, A. Scheidegger, The Physics of Flow Through Porous Media, Toronto: University of Toronto Press, 1974; R. S. Mikhail and E. Robens, Microstructure and Thermal Analysis of Solid Surfaces, Chichester: Wiley, 1983; F. Dullien, Porous Media—Fluid Transport and Pore Structure, San Diego: Academic Press, 1992; and K. Meyer et al., “Porous Solids and Their Characterization,” Crystal Research and Technology, (1994), vol. 29, p. 903, the contents of each of which are incorporated herein by reference.

To test cell seeding and growth, the circular sample scaffolds were transferred to the wells of a 96-well plate, and sterilized by immersion in 70% ethanol for at least 30 min, and by exposure to ultraviolet radiation for 15-30 min after ethanol removal. Under sterile conditions, the scaffolds were rinsed once with phosphate buffered saline and twice with tissue culture media, Dulbecco's Modified Eagle Medium (DMEM) containing 10% fetal bovine serum (FBS). HepG2 cells were trypsinized from confluent culture to obtain a cell suspension, and ˜10⁵ cells were seeded in each scaffold-containing well.

The test results show that sample scaffolds formed from RGD-modified fibres are more amenable to cell attachment and proliferation than non-modified scaffolds. Good cell viability, however, was found with both modified and non-modified sample scaffolds. The collagen-modified scaffolds have tree-trunk-like morphology indicating incorporation of the collagen. The results demonstrate that the scaffolds formed according to exemplary embodiments of the present invention can serve as excellent tissue template and/or platform for presentation of biological signals to regulate cell adhesion and phenotype.

As now can be appreciated, advantageously, scaffolds formed as described herein are porous and can have high porosity and large pore sizes. Further, the exemplary processes described above do not require excessive heat exchange or addition of chemicals such as binders or stabilizers which could have adverse effects on the modifiers such as proteins incorporated into the fibres.

A further advantage of these exemplary processes is that impurities and other undesirable substances, such as molecules of low molecular weight, can be conveniently removed from the fibres by for example water while they are entangled to form the scaffold.

In addition, it is relatively easy to form scaffolds having different regional properties and characteristics by entangling different fibres together.

The scaffolds prepared as described above can have applications in many fields including tissue engineering, 3-D cell culturing, 3-D cell culture system for high-throughput drug screening, drug-releasing fabrics, containers for expansion of cells such as stem cells, and the like.

In this description, when the conditions for a reaction or process are not expressly provided, the conditions can be assumed to be the standard conditions and can vary within the range of normal conditions. In particular, the normal conditions may include standard conditions such as atmospheric pressure and room temperature.

Other features, benefits and advantages of the embodiments described herein not expressly mentioned above can be understood from this description and the drawings by those skilled in the art.

The contents of each reference cited above are hereby incorporated herein by reference.

Of course, the above described embodiments are intended to be illustrative only and in no way limiting. The described embodiments are susceptible to many modifications of form, arrangement of parts, details and order of operation. The invention, rather, is intended to encompass all such modification within its scope, as defined by the claims.

What is claimed is:

1. A method of forming a porous scaffold, comprising the steps of:
providing fibres comprising polyelectrolytes forming a polyelectrolyte complex, said fibres further comprising a cross-linker linking said polyelectrolytes within individual ones of said fibres for inhibiting secondary polyelectrolyte complexation between adjacent fibres; and
applying a fluid to said fibres to entangle said fibres to form a porous structure.

2. The method of claim 1, wherein said cross-linker comprises silicon.

3. The method of claim 2, wherein said cross-linker links said polyelectrolytes through Si—O bonds.

4. The method of claim 2, wherein said cross-linker comprises silica.

5. The method of claim 1, wherein said cross-linker is selected from acrylates, succinimides, carbodiimides, and quinones.

6. The method of claim 1, wherein said polyelectrolytes are selected from alginate, chitosan, chitin, heparin, chondroitin sulfate, hyaluronic acid, DNA, RNA, poly(ornithic acid), polyacrylic acid, poly(ethyleneimine), gellan, carboxylated polymer, aminated polymer, chitosan derivative, chitin derivative, acrylate polymer, nucleic acid, histone protein, acidic polysaccharide, derivative of acidic polysaccharide, poly(amino acid), poly(lysine), and poly(glutamic acid).

7. The method of claim 6, wherein said polyelectrolyte complex is selected from alginate-chitosan, heparin-chitosan, chondroitin sulfate-chitin, hyaluronic acid-chitosan, DNA-chitin, RNA-chitin, poly(glutamic acid)-poly(ornithic acid), polyacrylic acid-poly(lysine), and poly(ethyleneimine)-gellan complexes.

8. The method of claim 1, wherein said polyelectrolyte complex is an alginate-chitosan complex.

9. The method of claim 1, wherein said fibres are formed from a polyanion solution and a polycation solution by interfacial polyelectrolyte complexation, said polyanion solution comprising a polyanion and said polycation solution comprising a polycation.

10. The method of claim 9, wherein said polyanion solution comprises alginate.

11. The method of claim 9, wherein at least one of said polyanion and polycation solutions comprises at least one of said cross-linker and a precursor of said cross-linker.

12. The method of claim 11, wherein said polycation solution comprises said precursor.

13. The method of claim 11, wherein said precursor comprises hydrolyzed tetraethyl orthosilicate (TEOS).

14. The method of claim 9, wherein said polycation solution comprises chitosan.

15. The method of claim 9, wherein said polycation solution comprises chitosan and hydrolyzed tetraethyl orthosilicate (TEOS), the weight ratio of said chitosan and TEOS being between 8:0 and 1:19.

16. The method of claim 15, wherein said weight ratio is from 8:3.7 to 1:9.4.

17. The method of claim 9, wherein said step of providing fibres comprises bringing said polyanion and polycation solutions into contact to form an interfacial region, and drawing said fibres from said interfacial region.

18. The method of claim 17, wherein said interfacial region comprises chitosan and alginate with a weight ratio from 8:1 to 1:16.

19. The method of claim 1, wherein said fibres further comprise a modifier for modifying a property of said fibres.

20. The method of claim 19, wherein said modifier comprises a surface-modifying substance.

21. The method of claim 19, wherein said modifier comprises at least one of a protein and a peptide.

22. The method of claim 19, wherein said modifier comprises at least one of polyethylene glycol (PEG), collagen, and a peptide with an arginine-glycine-aspartate (RGD) motif.

23. The method of claim 1, wherein said fibres are confined in a die during said step of applying a fluid such that said porous structure has an external profile substantially conforming to an inner surface of said die.

24. The method of claim 1, wherein said fluid comprises water.


SEQUENCE LISTING (1 sequence)

SEQ ID NO: 1 - protein, 11 aa, artificial
  synthetic peptide sequence
GCGYGRGDSPG